SEQUENCE LISTING (12 sequences)

SEQ ID NO: 1 - DNA, 20 bp, Artificial Sequence
  Actin forward primer
caatgtggccgaggactttg

SEQ ID NO: 2 - DNA, 21 bp, Artificial Sequence
  Actin reverse primer
cattctccttagagagaagtg

SEQ ID NO: 3 - DNA, 20 bp, Artificial Sequence
  Nkx2.5 forward primer
agaagacagaggcggacaac

SEQ ID NO: 4 - DNA, 18 bp, Artificial Sequence
  Nkx2.5 reverse primer
cgccgctccagttcacag

SEQ ID NO: 5 - DNA, 20 bp, Artificial Sequence
  alpha-MHC forward primer
attgctgaaaccgagaatgg

SEQ ID NO: 6 - DNA, 20 bp, Artificial Sequence
  alpha-MHC reverse primer
cgctccttgaggttgaaaag

SEQ ID NO: 7 - DNA, 20 bp, Artificial Sequence
  NeuroD forward primer
gccccagggttatgagacta

SEQ ID NO: 8 - DNA, 19 bp, Artificial Sequence
  NeuroD reverse primer
gtccagcttggaggacctt

SEQ ID NO: 9 - DNA, 20 bp, Artificial Sequence
  Oct4 forward primer
ggcaacctggagaatttgtt

SEQ ID NO: 10 - DNA, 20 bp, Artificial Sequence
  Oct4 reverse primer
gccggttacagaaccacact

SEQ ID NO: 11 - DNA, 20 bp, Artificial Sequence
  AFP forward primer
gtagcgctgcaaacaatgaa

SEQ ID NO: 12 - DNA, 20 bp, Artificial Sequence
  AFP reverse primer
tccaacaggcctgagaaatc

(19) United States
(12) Patent Application Publication
(10) Pub. No.: US 2010/0247493 A1
(43) Pub. Date: Sep. 30, 2010

(54) Method for Identifying and Selecting Cardiomyocytes

(76) Inventors: William L. Rust, Singapore (SG); Thavamalar Balakrishnan, Singapore (SG)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/671,274

(22) PCT Filed: Jul. 31, 2007
(86) PCT No.: PCT/SG07/00226

§ 371 Date: Jan. 29, 2010

(51) Int. Cl. A61K 35/12; G01N 33/53; C12N 5/071; A61P 9/00

(52) U.S. Cl. 424/93.7; 435/7.21; 435/325

(57) ABSTRACT

The present invention relates to new and/or improved methods of identification and selection of cardiomyocytes from human embryonic stem (hES) cells. The method further comprises isolating the selected cardiomyocyte population. There is also provided method for the screening for cardiovascular compounds comprising subjecting the said cardiomyocyte population to test compound/s, and observing and/or interpreting a response of the cardiomyocytes to the test compound.

### FIELD OF THE INVENTION

The present invention relates to the identification and isolation of cardiomyocytes from human embryonic stem (hES) cells.

### BACKGROUND OF THE INVENTION

Cardiovascular diseases remain the leading cause of mortality and morbidity world wide. Since adult cardiomyocytes do not regenerate, the death of these cells compromises the myocardial contractile function. For instance when the coronary vessel is occluded by a thrombus and the surrounding cardiomyocytes cannot be supplied with necessary energy sources from other coronary vessels. The loss of functional cardiomyocytes may lead to chronic heart failure. A potential route of restoring normal heart function is replacement of injured and dead cardiomyocytes by new functional cardiomyocytes.

The success of regenerative cardiac medicine depends on the availability of cardiomyocytes in sufficient numbers for the transplantation of the cardiac tissue. Cardiomyocytes have the potential to restore heart function after myocardial infarction or heart failure and human embryonic stem (hES) cells are potential source of transplantable cardiomyocytes (Siu et al, 2007).

A limitation in the study of cardiomyocytes has been the inability to identify these cells prospectively. The current protocols designed to direct the differentiation of human embryonic stem cells in vitro towards cells of the cardiomyocyte lineage produce a heterogeneous population of cells of various identity and developmental stage. For the purpose of producing cell therapies or diagnostic cell products, pure or relatively pure cardiomyocyte populations are desired. Nearly pure populations of cardiomyocytes have been generated from mouse embryonic stem cells using a method requiring prior genetic transformation of the stem cells. Genetic transformation of stem cells is time consuming and may preclude the enriched cell population from use in the clinic. It would therefore be an advantage to have a method capable of isolating a population of differentiated cells enriched for cardiomyocytes from wild-type stem cells. Furthermore, it would be an advantage if a large proportion of the enriched cardiomyocytes were viable and capable of proliferation, allowing the enriched population to expand in culture for a number of population doublings.

### SUMMARY OF THE INVENTION

The present invention addresses the problems above and in particular provides new and improved method of identification and isolation of cardiomyocytes from differentiated embryonic stem (ES) cells.

According to a first aspect, the present invention provides a method of identifying and selecting a cardiomyocyte population from a heterogeneous population of differentiated stem cells, comprising contacting the heterogeneous cell population with at least one agent that specifically binds to at least one cardiomyocyte marker and selecting the bound cells as cardiomyocytes.

The method further comprises isolating the selected cardiomyocyte population. There is also provided a method of propagating the selected cardiomyocyte population in culture. In particular, the at least one cardiomyocyte marker is selected from a group consisting of CD166 (ALCAM), VEGF receptor Flk1, N-cadherin, CD133 and CD117 (C-kit). More in particular the at least one cardiomyocyte marker is CD166 (ALCAM). The at least one cardiomyocyte marker may be a fetal marker. The identified cells may comprise at least 50% cardiomyocytes. In particular the identified cardiomyocytes may have a fetal phenotype. For example cardiomyocytes may be capable of proliferating in culture. In particular at least 25% of the identified cardiomyocytes may be in S phase of the cell cycle. More in particular the identified cardiomyocytes are capable of rhythmic contractions and/or forming electrically coupled cell clusters. As a non-limitative example, the stem cells may be selected from a group consisting of embryonic stem (ES) cell, pluripotent stem cells, hematopoietic stem cells, totipotent stem cells, mesenchymal stem cells, neural stem cells and adult stem cells. In particular the stem cells may be human ES cells.

According to another aspect, the invention provides a cardiomyocyte population having the characteristics as herein defined. In particular, there is provided a cardiomyocyte population identified and/or isolated by the method according to the present invention. There is also provided a cardiomyocyte population isolated according to the method of the present invention.

According to yet another aspect, the invention provides a model for study of human cardiomyocytes in culture, comprising the cardiomyocyte population. The invention further provides a kit for cardiotoxic testing comprising the cardiomyocyte population.

Another aspect of the invention includes a method of preventing, repairing and/or treating at least one cardiac disorder in a subject, the said method comprising transplanting the isolated cardiomyocyte population. The cardiac disorder may be selected from a group consisting of myocardial infarction, cardiomyopathy, congestive heart failure, ventricular septal defect, atria septal defect, congenital heart defect and ventricular aneurysm.

According to a further aspect, the invention provides a model for testing suitability of cardiomyocytes for cardiac transplantation, said model comprising:
A non-human animal having a measurable parameter of cardiac function wherein the said animal is capable of receiving an isolated cardiomyocyte population; and
a means to determine cardiac function of the animal before and after transplantation of the isolated cardiomyocyte population. In particular the model may be an immunodeficient animal created as a model of cardiac muscle degeneration following infarct that is used as a universal acceptor of the isolated cardiomyocyte population. More in particular the animal model may be murine, ovine, bovine, porcine or a non-human primate. More in particular the parameter of cardiac function may be contractile function.

According to yet another aspect the invention provides a method of screening for cardiovascular compounds. In particular the method may comprise subjecting the said cardiomyocyte population to at least one test compound, and observing a cardiac specific response of the cardiomyocytes to at least one test compound. In particular the cardiac specific response may comprise alteration of Q-T wave.

### BRIEF DESCRIPTION OF THE FIGURES

FIG. 1 represents the expression of the cardiac transcription factor Nkx2.5 analysed by immunofluorescence following culturing of human embryonic stem cells for 14 days, under conditions which promote cardiomyocyte differentiation. The nuclei are counterstained with DAPI (blue), the area shown by arrows.

FIG. 1A represents the co-localization of Nkx2.5 (green) with the cardiac marker αMHC (red). The black and white view of FIG. 1A represents the co-localization of Nkx2.5 (dark white) with the cardiac marker αMHC (light grey)

FIG. 1B represents the co-localization of Nkx2.5 (red) with the cardiac marker MLC2a (green). The black and white view of FIG. 1B represents the co-localization of Nkx2.5 (light grey) with the cardiac marker MLC2a (dark white).

FIG. 1C represents the co-localization of Nkx2.5 (red) with the cardiac marker alpha-actinin (green). The black and white view of FIG. 1C represents the co-localization of Nkx2.5 (light grey) with the cardiac marker alpha-actinin (dark white).

FIG. 1D represents the co-localization of Nkx2.5 (red) with the cardiac marker tropomyosin (green). The black and white view of FIG. 1D represents the co-localization of Nkx2.5 (light grey) with the cardiac marker tropomyosin (dark white).

FIG. 1E represents the co-localization of Nkx2.5 (red) with the cardiac marker MLC2v (green). The black and white view of FIG. 1E represents the co-localization of Nkx2.5 (light grey) with the cardiac marker MLC2v (dark white).

FIG. 2 represents the expression of the cardiac transcription factor Nkx2.5 (green) analysed by immunofluorescence following culturing of human embryonic stem cells for 14 days under conditions which promote cardiomyocyte differentiation. The nuclei are counterstained with DAPI (blue), the area shown by arrows.

FIG. 2A represents the co-localization of Nkx2.5 (green) with the cardiac marker CD166 (red). The black and white view of FIG. 2A represents the co-localization of Nkx2.5 (dark white) with the cardiac marker CD166 (light grey).

FIG. 2B represents the co-localization of Nkx2.5 (green) with the cardiac marker Flk-1 (red). The black and white view of FIG. 2B represents the co-localization of Nkx2.5 (dark white) with the cardiac marker Flk-1 (light grey).

FIG. 2C represents the co-localization of Nkx2.5 (green) with the cardiac marker N-cadherin (red). The black and white view of FIG. 2C represents the co-localization of Nkx2.5 (dark white) with the cardiac marker N-cadherin (light grey).

FIG. 3 represents percentage of surviving adherent cells at 48 hours, following digestion of the embryoid bodies and undifferentiated hES with trypsin or accumax reagent, and plating of the single cell suspensions on collagen I treated tissue culture dishes.

FIG. 4 represents quantitative PCR analysis of RNA extracted from MACS sorted cells based on expression of CD166. Figure represents the expression of the cardiac markers Nkx2.5 and αMHC, neural marker NeuroD1, pluripotent cells marker Oct4 and endodermal cells marker AFP on cells isolated based on expression of CD166 enriched for cardiomyocytes.

FIG. 5 represents proliferation of cells in collagen I coated culture dishes, isolated based on expression of CD166.

FIG. 5A represents the sub-confluent layer of surviving cells attached to the dish after one day in culture.

FIG. 5B represents confluent layer of surviving cells attached to the dish after six days in culture.

FIG. 5C represents the analysis of cells in S phase by BrdU (green) incorporation into the layer of surviving cells attached to the dish after two days in culture. In black and white view of FIG. 5C represents the analysis of cells in S phase by BrdU (dark grey) incorporation into the layer of surviving cells attached to the dish after two days in culture

FIG. 6 represents immunofluorescence analysis of the expression of Nkx2.5 (dark pink) and MLC2a (green) following the sorting of the 14 day old EBs based on expression of CD166, and plated on collagen I coated dishes in medium containing bovine serum and allowed to grow to confluence over a period of five days. Nuclei are counterstained with DAPI (blue). In the black and white view the Nk2.5 stained cells appear to be dark white while the DAPI counterstain appear as light grey.

FIG. 6A represents CD166+ cells expressing the cardiomyocyte marker Nkx2.5 (dark pink). The black and white view of FIG. 6A represents CD166+ cells expressing the cardiomyocyte marker Nkx2.5 (dark white).

FIG. 6B represents that CD166− cells do not express or express very little of the cardiomyocyte marker Nkx2.5 (dark pink). The black and white view FIG. 6B represents that CD166− cells do not express or express very little of the cardiomyocyte marker Nkx2.5 (dark white)

FIG. 6C represents CD166+ cells expressing the cardiomyocyte markers Nkx2.5 (dark pink) and MLC2a (green). The black and white view FIG. 6C represents CD166+ cells expressing the cardiomyocyte markers Nkx2.5 (dark white) and MLC2a (grey).

### BRIEF DESCRIPTION OF THE SEQUENCES

SEQ ID NO. 1 refers to Actin forward primer
5′-CAATGTGGCCGAGGACTTTG-3′
SEQ ID NO. 2 refers to Actin reverse primer
5′-CATTCTCCTTAGAGAGAAGTG-3′
SEQ ID NO. 3 refers to Nkx2.5 forward primer
5′-AGAAGACAGAGGCGGACAAC-3′
SEQ ID NO. 4 refers to Nkx2.5 reverse primer
5′-CGCCGCTCCAGTTCACAG-3′
SEQ ID NO. 5 refers to αMHC forward primer
5′-ATTGCTGAAACCGAGAATGG-3′
SEQ ID NO. 6 refers to αMHC reverse primer
5′-CGCTCCTTGAGGTTGAAAAG-3′
SEQ ID NO. 7 refers to NeuroD forward primer
5′-GCCCCAGGGTTATGAGACTA-3′
SEQ ID NO. 8 refers to NeuroD reverse primer
5′-GTCCAGCTTGGAGGACCTT-3′
SEQ ID NO. 9 refers to Oct4 forward primer
5′-GGCAACCTGGAGAATTTGTT-3′
SEQ ID NO. 10 refers to Oct4 reverse primer
5′-GCCGGTTACAGAACCACACT-3′
SEQ ID NO. 11 refers to AFP forward primer
5′-GTAGCGCTGCAAACAATGAA-3′
SEQ ID NO. 12 refers to AFP reverse primer
5′-TCCAACAGGCCTGAGAAATC-3′

### DETAILED DESCRIPTION OF THE INVENTION

Bibliographic references mentioned in the present specification are for convenience listed in the form of a list of references and added at the end of the examples. The whole content of such bibliographic references is herein incorporated by reference.

The present invention provides new and/or improved method of identification and isolation of cardiomyocytes from differentiated embryonic stem (ES) cells.

According to one aspect, the invention provides a method of identifying and selecting a cardiomyocyte population from a heterogeneous population of differentiated stem cells, comprising contacting the heterogeneous cell population with at least one agent that specifically binds to at least one cardiomyocyte marker and selecting cells bound to the said agent as cardiomyocytes. The heterogeneous population of differentiated stem cells may be prepared according to the method described in WO 2007/030870 (the content of which is herein incorporated by reference).

The method further comprises isolating the selected cardiomyocyte population. There is also provided a method of propagating the selected cardiomyocyte population in culture. In particular, the at least one cardiomyocyte marker is selected from the group consisting of CD166 (ALCAM), VEGF receptor Flk1, N-cadherin, CD133 and CD117 (C-kit). More in particular the at least one cardiomyocyte marker is CD166 (ALCAM). The at least one cardiomyocyte marker may be a fetal marker.

Sorting of cells based on surface marker expression may be accomplished by using any technology known in the art. For example, sorting of cells based on surface marker expression may be accomplished by using Flow Assisted Cell Sorting (FACS) or Automated Magnetic Cell Sorting (MACS) technology. The preparation of the cells for FACS is similar to preparation of cells for MACS except that the secondary antibody is conjugated to a FACS-compatible fluorophore instead of a magnetic microbead.

At least 50% of the identified, selected and/or isolated cells according to the invention may comprise cardiomyocytes. In particular, 55%, 60%, 70%, 80% or 90% of the isolated cells may comprise cardiomyocytes. In particular the identified, selected and/or isolated cardiomyocytes may have a fetal phenotype.

The cardiomyocytes may be capable of proliferating in culture. In particular at least 25% of the identified cardiomyocytes may be in S phase of the cell cycle.

More in particular, the identified cardiomyocytes are capable of rhythmic contractions and/or forming electrically coupled cell clusters.

As a non-limiting example, the stem cells may be selected from a group consisting of embryonic stem (ES) cell, pluripotent stem cells, hematopoitic stem cells, totipotent stem cells, mesenchymal stem cells, neural stem cells and adult stem cells. In particular the stem cells may be human ES cells. In particular the stem cells may be isolated ES cells. For example, the ES cell may be obtained from at least one ES cell line recognised the NIH human stem cell registry (http://stemcells.nih.gov/research/registry/defaultpage.asp) according to the methods and ethical standards mentioned therein. More in particular, the hES cell line hES3 from ES Cell International may be used.

“Stem cells” as described herein refers to a stem cell that is undifferentiated prior to culturing and is capable of undergoing differentiation. The stem cells may be selected from a group consisting of embryonic stem (ES) cell, pluripotent stem cells, hematopoietic stem cells, totipotent stem cells, mesenchymal stem cells, neural stem cells and adult stem cells. In particular the stem cell may be human embryonic stem (hES) cells. For example the stem cell may be derived from a cell culture, such as hES cells. The stem call may be derived from an embryonic cell line or embryonic tissue. The embryonic stem cells may be cells which have been cultured and maintained in an undifferentiated state.

The stem cells suitable for use in the present methods may be derived from a patient's own tissue. This would enhance compatibility of differentiated tissue grafts derived from stem cells with the patient.

Differentiated stem cells may express markers on their cell surface that may be indicative of a specific cell type, for example indicative of cardiomyocytes. The markers may be used to identify and isolate the differentiated cardiomyocytes from other differentiated cells and undifferentiated stem cells. “Markers”, as used herein, are polypeptide molecules that are expressed on a cell of interest. The specific marker may be present only in the cells of interest, or encompass the cells of interest, or detectable level of the marker is sufficiently higher in the cells of interest, compared to other cells, such that the cells of interest can be identified, using any of a variety of methods as known in the art. It will be understood by those of skill in the art that expression is a relative term, and the expression will vary from other cell types. For example, a progenitor cell may express a polypeptide that is not found in the fully differentiated progeny cell. A cell of interest may express a polypeptide that is not expressed in surrounding tissues, e.g. the cardiomyocyte cells of fetal phenotype may express CD166 polypeptides not found in mature cardiomyocytes or on other cells of a non-cardiomyocyte lineage. This specificity is sufficient for purposes of cell identification and isolation. Therefore, “fetal markers” as used herein refer to a marker on a cell, in particular cardiomyocytes that is indicative of the fetal phenotype of the cells. Fetal phenotype further refers to cells that are capable of proliferating in culture. Some fetal markers of interest in the present invention include CD166 (ALCAM), VEGF receptor Flk1, N-cadherin, CD133, CD117 (C-kit), Nkx2.5, α-MHC, MLC2a, MLC2v, α-actinin and tropomyosin. In particular, fetal markers of interest in the present invention include CD166 (ALCAM), VEGF receptor Flk1, N-cadherin, CD133, CD117 (C-kit). More in particular the cardiomyocyte marker may be CD166 (ALCAM). These markers are well known in the art, and agents (reagents) for the detection thereof are widely available. In a typical assay for detection and/or isolation, a heterogeneous population of differentiated stem cell is contacted with at least one a marker-specific “agent”, and detecting directly or indirectly the presence of the complex formed. The term “agent” as used herein refers to a molecule capable of binding to another molecule, for example the marker on the cell surface, through chemical or physical means, wherein the agent and the marker form a binding pair. For example antibodies specific for these cell surface markers are commercially available, or may be produced using conventional methods as known in the art, therefore the antibodies and markers form a binding pair.

Of particular interest is the use of antibodies as affinity reagents. Conveniently, these antibodies are conjugated with a label for use in separation. Labels include magnetic beads, which allow for direct separation on magnetic assisted cell sorter (MACS), biotin, which can be removed with avidin or streptavidin bound to a support, fluorochromes, which can be used with a fluorescence activated cell sorter (FACS), or the like, to allow for ease of separation of the particular cell type. Fluorochromes that find use include phycobiliproteins, e.g. phycoerythrin and allophycocyanins, fluorescein and Texas red. Frequently each antibody is labeled with a different fluorochrome, to permit independent analysis or sorting for each marker. Monoclonal antibodies specific for the markers may be produced in accordance with conventional ways, immunization of a mammalian host, e.g. mouse, rat, guinea pig, cat, dog, etc., fusion of resulting splenocytes with a fusion partner for immortalization and screening for antibodies having the desired affinity to provide monoclonal antibodies having a particular specificity. These antibodies can be used for affinity chromatography, ELISA, RIA, and the like. The antibodies may be labelled with radioisotopes, enzymes, fluorescers, chemiluminescers, or other label which will allow for detection of complex formation between the labelled antibody and its complementary epitope.

In particular the invention provides methods of preventing, repairing and/or treating at least one cardiac disorder in a subject, the method comprising transplanting the cardiomyocyte population in a subject. The subject is, in particular, a subject in need of the treatment thereof. The disorder as, used herein, include but are not limited to myocardial infarction, cardiomyopathy, congestive heart failure, ventricular septal defect, atria septal defect, congenital heart defect and ventricular aneurysm. In this aspect of the invention, the method includes introducing a cardiomyocyte population of the invention into cardiac tissue of a subject. In particular the isolated cardiomyocyte population is transplanted into damaged cardiac tissue of the subject. More in particular the method results in the restoration of cardiac function in a subject. The cardiomyocyte population may resemble a human fetal atrial cell in culture. In particular the cardiomyocyte population may resemble a human fetal pacemaker cell in culture. More in particular the cardiomyocyte population may comprise plurality of isolated cardiomyocytes wherein the cardiomyocytes may be coupled. The coupling may be, for example, through gap junctions and/or adherens junctions, wherein the coupling is electrical. The subject may be a human or non-human animal.

The present invention also provides at least one cardiomyocyte population identified, selected and/or isolated according to the method of the present invention for use in medicine. In particular, in preventing, repairing and/or treating at least one cardiac disorder in a subject. There is also provided the use of at least one cardiomyocyte population identified, selected and/or isolated according to the method of the present invention for the preparation of a medicament in preventing, repairing and/or treating at least one cardiac disorder in a subject.

The present invention also provides a cardiac model for testing the ability of the isolated cardiomyocyte population to restore cardiomyocyte function. In order to test the effectiveness of transplanted cardiomyocyte population in vivo, it is important to have a reproducible animal model with a measurable parameter of cardiac function. The parameters used should clearly distinguish control and experimental animals so that the effects of the transplantation can be adequately determined. A host animal, such as, but not limited to, an immunodeficient mouse may be used as a ‘universal acceptor’ of cardiomyocytes produced by the methods of the present invention.

The myocardial model of the present invention is designed to assess the extent of cardiac repair following transplant of cardiomyocytes into the host animal. In particular, the host animal may be an immunodeficient animal created as a model of cardiac muscle degeneration following infarct that is used as a universal acceptor of isolated cardiomyocytes. The non-human animal may be any species including but not limited to murine, ovine, canine, bovine, porcine and any non-human primates. Parameters used to measure cardiac repair in these animals may include, but are not limited to, electrophysiological characteristic of heart tissue or various heart functions. For instance, contractile function may be assessed in terms of volume and pressure changes in a heart. Methods of assessing heart function and cardiac tissue characteristics may also involve techniques known to person skilled in the art.

The invention further provides cardiomyocytes produced using the methods of the current invention that may be used for transplantation, cell therapy or gene therapy. In particular the invention provides the use of cardiomyocytes produced using the methods of the current invention, in a cardiac model for testing the ability to restore cardiac function. More in particular the invention provides the use of cardiomyocytes in a cardiac model designed to assess the extent of cardiac repair following transplant of cardiomyocytes into a suitable host animal.

The present invention also provides a model for study of human cardiomyocytes in culture, comprising the cardiomyocytes isolated by the method of the current invention. This model may be used in the development of cardiomyocyte transplantation therapies.

According to yet another aspect the invention provides a method of screening for cardiovascular compounds. In particular the method may comprise subjecting the said cardiomyocyte population to at least one test compound, and observing a cardiac specific response of the cardiomyocytes to at least one test compound. In particular, the specific cardiac response may be monitored by the changes of beat frequency, amplitude and/or duration of the cardiomyocyte(s) to at least one test compound. More in particular the cardiac specific response may comprise alteration of Q-T wave.

There is also provided a kit for cardiotoxic testing or for screening of cardiovascular compound(s) comprising at least one cardiomyocyte population according to the invention. There is also provided a kit for preventing, repairing and/or treating at least one cardiac disorder in a subject, the kit comprising at least one cardiomyocyte population according to the invention. The kit may further comprise instructions for use.

Having now generally described the invention, the same will be more readily understood through reference to the following examples which are provided by way of illustration, and are not intended to be limiting of the present invention.

### EXAMPLES

### Materials and Methods

### hES Cell Culture

The hES cell line hES3 from ES Cell International (http://stemcells.nih.gov/research/registry/esci.asp) were maintained on human fibroblasts in KO-DMEM with 20% KOSR in 0.1 mM beta-mercaptoethoethanal, 1% MEM non-essential amino acids, 2 mM L-glutamine, bFGF (10 ng/ml) with or without antibiotics (Penicillin/Streptomycin; all reagents from Invitrogen). The hES cells were passaged by treatment with collagenase I (however, collagenase IV may also be used) (Gibco) for 3 minutes followed by mechanical dissociation. Harvested cells were transferred to newly prepared feeder cells.

### hES Differentiation

The pluripotent hES grown on human feeders in 10 cm dishes were rinsed with phosphate buffered saline (PBS). PBS was then replaced by fresh stem cell maintenance medium. The dish was scored using a pipette tip such that each colony was divided approximately in two cell clusters. Cell clusters were scraped from the substrate and transferred to a conical tube. The cell clusters were allowed to settle to the bottom of the conical tube and the media was aspirated. The media was replaced by fresh stem cell maintenance medium. The cell clusters were transferred to plastic dishes to discourage cell attachment (Ultra-low attach dishes, Costar). The dishes were incubated in the tissue culture incubators for a period of 24 hours. After the 24 hour culture, embryoid bodies (EBs) were formed from the pluripotent cell clusters in suspension. The dishes were tilted such that the embryoid bodies sank to the bottom and the media was aspirated. The medium was replaced by defined basic serum free (bSFS) medium comprising DMEM supplemented with 1×MEM non-essential amino acids (Invitrogen), 2 mM L-Glutamine (Invitrogen), 0.0055 mg/ml Transferrin (Roche), 5 ng/ml sodium Selenite (Sigma), 0.1 mM beta-mercaptoethanol, with or without Penicillin/Streptomycin (Invitrogen). which promotes cardiomyocyte differentiation as described in WO 2007/030870.

To further encourage differentiation towards the cardiomyocyte lineage, 5 μg/ml of the compound SB203580, as described in WO 2007/030870, was added. The embryoid bodies were cultured in these conditions for an additional 12 days. During this period, the culture medium was changed every 3-4 days.

### Digestion of Embryoid Bodies to a Single Cell Suspension.

The EBs were transferred to a conical tube and allowed to settle. The medium was aspirated and the EBs rinsed with PBS not containing either magnesium or calcium. EBs were incubated at 37° C. in either undiluted Accumax reagent (Innovative Cell Technologies), or a 0.25 or 0.005% solution of trypsin (Roche) in phosphate buffered saline. The enzymatic reactions were arrested by the addition of differentiation medium containing 20% fetal calf serum. Residual clusters of cells were removed by passing the cell suspension through a filter with maximum pore size of 40 μm.

### Magnet Assisted Cells Sorting (MACS)

The single cell suspensions were pelleted in a centrifuge refrigerated to 4° C. at approximately 300 gravities for 15 minutes. The cell pellet was resuspended in an immunoglobulin blocking buffer (FcR blocking buffer, Miltenyi Biotec) at a concentration of 1×10⁶ cells per 1000. A concentration of 0.5 to 5 μg/ml of antibody (mouse monoclonal ab23829, Abcam) which binds the cell surface antigen CD166 was added to the cell suspension. The cell suspension was incubated for 30 minutes at 4° C. while rocking. The cells were pelleted again in a refrigerated centrifuge at approximately 300 gravities for 10 minutes. The blocking buffer containing the anti-CD166 antibody was aspirated and replaced by 80 ul per 1×10⁶ cells supplemented with 20 ul of magnetic microbead conjugated antibody which recognizes the anti-CD166 antibody (rat anti-mouse igG2a+b microbeads, 472-01 Miltenyi Biotec) and was incubated for 30 minutes at 4° C. while rocking. The cells were pelleted and resuspended in fresh blocking buffer. Cells bound to magnetic microbeads were separated from the unbound cell population by being passed through a column held in a strong magnetic field (Miltenyi Biotec columns, Miltenyi Biotec magnetic holder). The sorted cells were pelleted, resuspended in bSFS media containing 5 μM SB203580 and 20% fetal calf serum and plated in tissue culture dishes pre-coated with 100 μg/ml of collagen I (Roche). The media was changed every 2-3 days. After the cultures had grown to confluence, the medium was replaced by bSFS medium containing 5 μM SB203580 but without fetal calf serum.

### Quantitative PCR

Total RNA was isolated using the RNeasy kit (Qiagen), treated with on-filter DNase and quantified by UV absorption. One μg of RNA was converted to cDNA using M-MuLV reverse transcriptase (New England Biolabs) using random hexamer primers and following manufacturer's instructions. Quantitative PCR was performed with 50 ng of each reverse transcriptase reaction, 250 nM of forward and reverse primer, 1×SYBR green PCR master mix (Bio-RAD) and analyzed by iCycler thermocycler (Bio-RAD). Primers comprising the sequence of SEQ ID NO:1 and SEQ ID NO:2 were used to detect binding amplification of the actin sequence, primers comprising the sequence of SEQ ID NO: 3 and SEQ ID NO:4 were used to detect Nkx2.5, primers comprising the sequence of SEQ ID NO:5 and SEQ ID NO:6 were used to detect αMHC sequence, primers comprising the sequence of SEQ ID NO: 7 and SEQ ID NO: 8 were used to NeuroD, primers comprising the sequence of SEQ ID NO. 9 and SEQ ID NO: 10 were used to amplify oct4 sequence and primers comprising the sequence of SEQ ID NO. 11 and SEQ ID NO: 12 were used to amplify AFP sequence. Expression was calculated based on a standard curve and normalized to II actin.

### Immunofluorescence

The EBs were fixed in 4% paraformaldehyde, cryo-preserved in 25% sucrose at 4° C. overnight, snap frozen in OCT media (Leica), and sectioned to 6 μm using a cryotome (Leica CM3050S). Sections were rinsed in PBS, fixed in 4% paraformaldehyde, permeabilized with 0.1% triton X-100 in PBS, incubated in block buffer (PBS, 0.1% Triton X-100, 1% BSA) and incubated overnight at 4° C. in block buffer containing primary antibodies against Nkx2.5 (1:200 dilution, Santa Cruz), αMHC (1:100 dilution Santa Cruz), MLC2a (1:500 dilution, Chemicon), MLC2v (1:500 dilution, Chemicon), Tropomyosin (1:50 dilution, Iowa Developmental Studies Hybridoma Bank), or alpha-actinin (1:50 dilution, Chemicon). After three rinses in PBS, slides were incubated for one hour at room temperature in blocking buffer containing secondary antibodies (1:1000 dilution, Chemicon, Zymed), incubated for one hour at room temperature, rinsed three times in PBS, incubated in DAPI (1:2000 dilution) for 10 minutes, rinsed, and mounted with Fluorosave (Calbiochem).

### BrdU Incorporation

Determination of cell proliferation was performed using in situ Cell Proliferation Kit, FLUOS (Roche) and following manufacturer's instructions. Briefly, 10 μM BrdU was added to the cell medium for a period of one or three hours. Cells were then fixed for immunohistochemistry and DNA was denatured by 20 min incubation in 4M HCl.

### Results

### Differentiation to Cardiomyocyte Lineage

Stem cells were stimulated to differentiate towards the cardiomyocyte lineage following the methods described in WO 2007/030870. At the end of this culture period, lasting two weeks, clusters of cells in suspension, termed embryoid bodies (EBs) were produced. A large proportion of the EBs began spontaneous rhythmic contractions and contained cells which expressed markers of the cardiomyocyte lineage.

Differentiated EBs Contain Cells which Express the Cardiomyocyte Transcription Factor Nkx2.5 and Cardiac Structural Proteins.

A highly specific and early marker of cardiac cell identity is the transcription factor Nkx2.5. The Nkx2.5 marker is expressed ubiquitously in all mouse heart cell progenitors around the time the heart crescent is formed and is an important regulator of cardiac gene expression in the developing and adult animals in both mice and humans (McFadden et al, 2002).

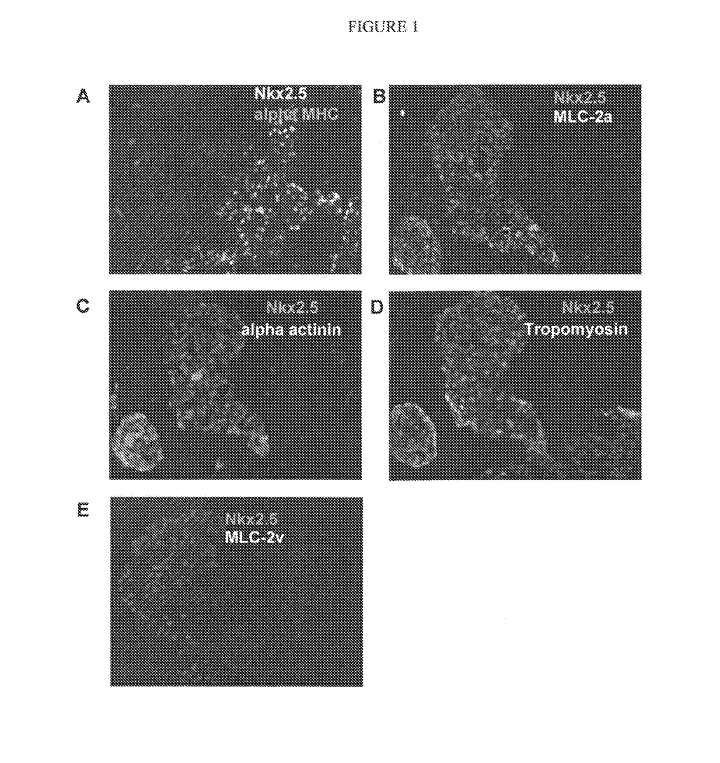

The marker Nkx2.5 was detected by immunofluorescence in cells of EBs differentiated according to the above protocol. Structural markers of the cardiac contractile machinery expressed in fetal cardiomyocytes were co-expressed in cells expressing Nkx2.5, confirming their cardiac identity (FIG. 1). It is known that αMHC and MLC2a are expressed throughout the myocardium in the developing mouse heart (Somi at al, 2006; Cai et al, 2005). FIG. 1A and FIG. 1B show that αMHC and MLC2a were co-expressed by clusters of cells which expressed Nkx2.5. Further since alpha actinin and tropomyosin are expressed in all cardiac contractile tissue, the co-expression of these markers by cells which expressed Nkx2.5 was seen as shown in FIG. 1C and FIG. 1D. It is further known that MLC2v expression is restricted to ventricle and atrioventricular canal when specification of these structures occurs (Cai et al, 2005). Accordingly MLC2v was not detected in differentiated EBs, suggesting that these cells are homologous to a fetal developmental stage wherein ventricular specification had not yet occurred (FIG. 1E).

Cardiomyocytes co-express surface markers useful for antibody-based cell selection.

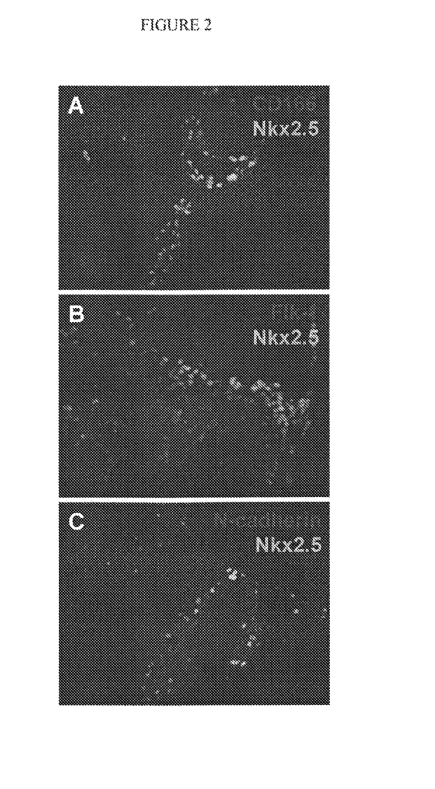

The surface marker CD166 (ALCAM) is an adhesive molecule expressed in the cardiac crescent and neural groove during mouse embryogenesis, and is lost in heart tissue by the time the mature heart has formed (Hirata et al, 2006). Therefore, cells isolated by expression of CD166 are likely to be developmentally immature and have the capacity to replicate in culture. In this study CD166 was co-expressed with Nkx2.5 by cells in the differentiated EBs, suggesting a fetal developmental stage of these cells (FIG. 2A). The VEGF receptor Flk-1 is expressed by mouse cardiac progenitors and is shown to be expressed in mouse embryonic stem cells with potential to differentiate to beating cardiomyocytes (Moretti et al, 2006; Kattman et al, 2006). Accordingly Nkx2.5 expressing cells in the EBs of the present invention was shown to co-express Flk-1 (FIG. 2B). Further N-cadherin is expressed continuously during heart development, and is associated with cardiac progenitor cells isolated from differentiating mouse embryonic stem cells (Honda et al, 2006). Accordingly Nkx2.5 expressing cells in the EBs also co-expressed the cell-cell adhesion molecule N-cadherin, (FIG. 2C).

### Isolation of Cell Population Enriched for Cardiomyoctes

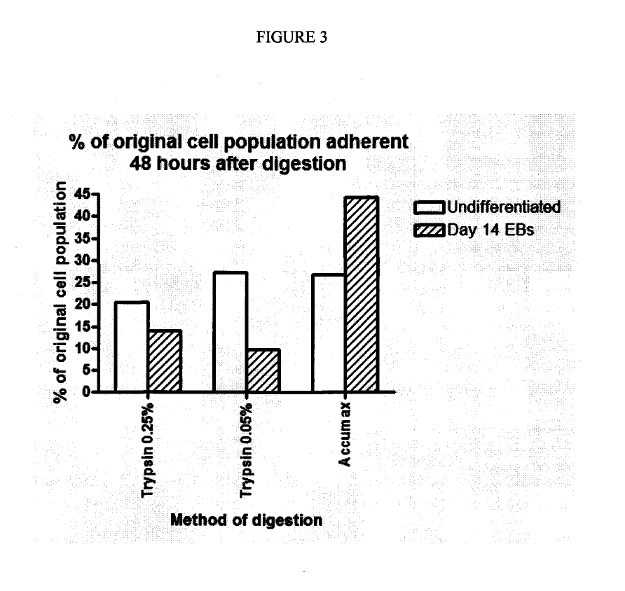

A single cell suspension prepared from differentiated EBs by gentle digestion with Accumax reagent was shown to survive better than that when digested with trypsin, and better than undifferentiated human embryonic stem cells digested by either method as shown in FIG. 3. Approximately 40% of differentiated cells digested using Accumax were capable of adhering to a tissue culture dish and remaining viable for at least 48 hours (FIG. 3). Subpopulations expressing the adhesion molecule CD166 were isolated from single cell suspensions by MACS as described in the materials and methods.

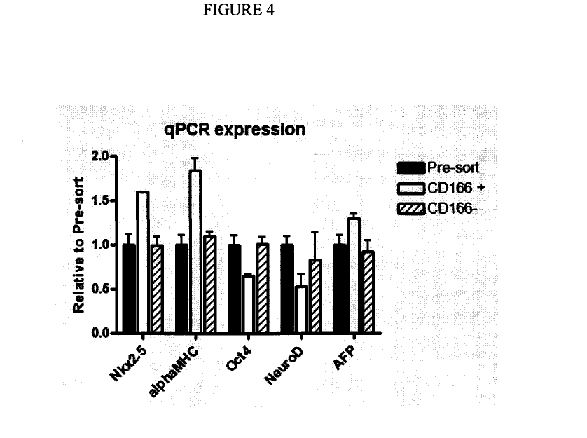

RNA extracted from cells immediately after sorting showed higher relative quantities of Nkx2.5 and αMHC transcripts in the CD166 expressing population than the CD166 negative or non-sorted populations (FIG. 4). In addition, cells sorted based on expression of CD166 have fewer transcripts of the neural marker NeuroD1 and the pluripotency marker Oct4. Therefore the sorted cardiomyocyte population of the current invention is depleted of non-cardiac cell types, including residual cells which presumably have the potential to form teratomas upon transplantation to a living animal.

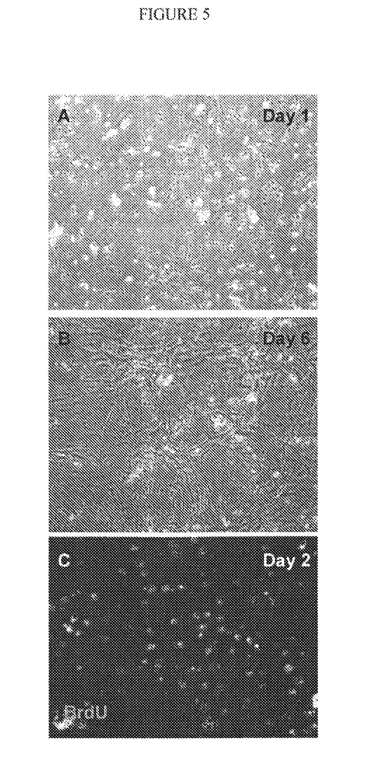

Although only a small proportion of the starting differentiated cell population may express CD166, one of the key features of the cells isolated based on expression of CD166 is that the cells are capable of replication in culture. The sorted cells selected by this method have the ability to grow rapidly in culture when plated at sub-confluent density. CD166− selected cells plated at approximately 30% confluence (FIG. 5A) in tissue culture dishes coated with collagen I in medium containing 5-20% fetal calf serum were able to grow to 100% confluence in culture within 6 days (FIG. 5B). Further during this growth phase it was seen 48 hours after plating that, approximately 25% of all adherent cells were in S-phase of the cell cycle as measured by BrdU incorporation (FIG. 5C). Alternatively, other adhesive substrates such as fibronectin can be used to stimulate cardiomyocyte attachment to the tissue culture dish. In addition, the use of bovine serum can be circumvented by the addition of growth-stimulating factors in the medium such as fibroblast growth factor or vascular endothelial growth factor.

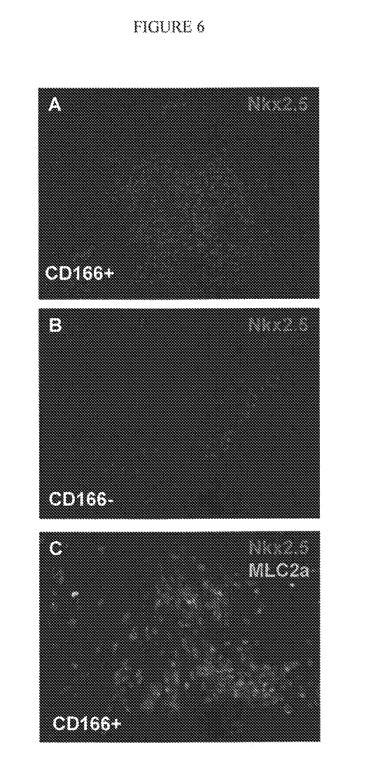

It is important that the cells isolated by the method of the current invention retain their cardiac identity and have the potential to form functional, electrically coupled cardiomyocytes. It was seen that populations of cells selected by expression of CD166 grown to confluence, begin spontaneous contractions, implying the presence of electrically coupled, functional cardiomyocytes. When these cells were fixed and visualized for expression of the cardiac marker Nkx2.5 by immunofluorescence, large clusters of cells expressed Nkx2.5. Visual count of representative fields of Nkx2.5 expressing cells revealed that cardiomyocytes represented greater than 50% of the total cell population (FIG. 6A). However cells that were negative for CD166 marker showed only basal level expression of Nkx2.5 (FIG. 6B). Cells expressing Nkx2.5 also co-expressed the cardiac structural marker myosin light chain 2a (MLC2a) confirming their cardiac identity as shown in FIG. 6C.

All the above experiments were performed with stringent controls. The results from the experiments suggest that identification and isolation of cardiomyocytes based on the expression of CD166 marker is an efficient way of obtaining a population of cells enriched in cardiomyocytes. Further, in addition to the surface marker CD166, selection of cells can be based on expression of other markers including Flk1, N-cadherin, CD133, and CD117. Further in addition to MACS, sorting of cells based on surface marker expression can be accomplished equally as well using other methods known to those skilled in the art, for example, FACS.

Finally, the invention as described herein is susceptible to variations, modifications and/or additions other than those specifically described and it is understood that the invention includes all such variations, modifications and/or additions which fall within the scope of the description as described herein.

### REFERENCES

Cai C L, Zhou W, Yang L, Bu L, Qyang Y, Zhang X, Li X, Rosenfeld M G, Chen J, Evans S., 2005. T-box genes coordinate regional rates of proliferation and regional specification during cardiogenesis. Development. 132(10):2475-87.
Hirata H, Murakami Y, Miyamoto Y, Tosaka M, Inoue K, Nagahashi A, Jakt L M, Asahara T, Iwata H, Sawa Y, Kawamata S., 2006 ALCAM (CD166) is a surface marker for early murine cardiomyocytes. Cells Tissues Organs. 184(3-4):172-80.
Honda M, Kurisaki A, Ohnuma K, Okochi H, Hamazaki T S, Asashima M., 2006. N-cadherin is a useful marker for the progenitor of cardiomyocytes differentiated from mouse ES cells in serum-free condition. Biochem Biophys Res Commun. 351(4):877-82.
Kattman S J, Huber T L, Keller G M., 2006. Multipotent flk-1+ cardiovascular progenitor cells give rise to the cardiomyocyte, endothelial, and vascular smooth muscle lineages. Dev Cell. 11(5):723-32.
McFadden D G, Olson E N., 2002. Heart development: learning from mistakes. Curr Opin Genet Dev. 12(3):328-35. Review.
Moretti A, Caron L, Nakano A, Lam J T, Bernshausen A, Chen Y, Qyang Y, Bu L, Sasaki M, Martin-Puig S, Sun Y, Evans S M, Laugwitz K L, Chien K R., 2006. Multipotent embryonic isl1+ progenitor cells lead to cardiac, smooth muscle, and endothelial cell diversification. Cell. 127(6):1151-65
Siu C W, Moore J C, Li R A., 2007. Human embryonic stem cell-derived cardiomyocytes for heart therapies. Cardiovasc Hematol Disord Drug Targets. 7(2):145-52.
Somi S, Klein A T, Houweling A C, Ruijter J M, Buffing A A, Moorman A F, van den Hoff M J., 2006. Atrial and ventricular myosin heavy-chain expression in the developing chicken heart: strengths and limitations of non-radioactive in situ hybridization. J Histochem Cytochem. 54(6):649-64.
WO 2007/030870.

1. A method of identifying and selecting a cardiomyocyte population from a heterogeneous population of differentiated stem cells, comprising contacting the heterogeneous cell population with at least one agent that specifically binds to at least one cardiomyocyte marker and selecting cells bound to the said agent as cardiomyocytes.

2. The method according to claim 1, further comprising a step of isolating the selected cardiomyocyte population.

3. The method according to claim 2, further comprising a step of propagating the selected cardiomyocyte population in culture.

4. The method according to claim 1, wherein the cardiomyocyte marker is selected from a group consisting of CD166 (ALCAM), VEGF receptor Flk1, N-cadherin, CD133 and CD117 (C-kit).

5. The method according to claim 1, wherein the cardiomyocyte marker is CD166 (ALCAM).

6. The method according to claim 1, wherein the cardiomyocyte marker is a fetal marker.

7. The method according to claim 2, wherein at least 50% of the isolated cells comprise cardiomyocytes.

8. The method according to claim 1, wherein the identified cardiomyocytes have a fetal phenotype.

9. The method according to claim 1, wherein the identified cardiomyocytes are capable of proliferating in culture.

10. The method according to claim 1, wherein the identified cardiomyocytes are capable of rhythmic contractions and optionally form electrically coupled cell clusters.

11. The method according to claim 1, wherein the stem cells are selected from the group consisting of embryonic stem (ES) cell, pluripotent stem cells, hematopoietic stem cells, totipotent stem cells, mesenchymal stem cells, neural stem cells and adult stem cells.

12. The method according to claim 11, wherein the stem cells are human ES cells.

13. (canceled)

14. A cardiomyocyte population, isolated by the method according to claim 2.

15. (canceled)

16. A kit for cardiotoxic testing comprising the cardiomyocyte(s) according to claim 14.

17. A method of preventing, repairing or treating at least one cardiac disorder in a subject, the said method comprising transplanting the cardiomyocyte population isolated and/or enriched according to claim 14.

18. (canceled)

19. A method of screening for cardiovascular compounds useful for modulating cardiac cell functions, said method comprising contacting a cardiomyocyte population according to claim 14 with at least one compound, and determining whether said compound produces a cardiac specific response in the cardiomyocytes relative to untreated cardiomyoctes.

20. The method according to claim 19, wherein the cardiac specific response comprises alteration of the Q-T wave.
